(19) United States
(12) Patent Application Publication
(10) Pub. No.: US 2025/0213749 A1
(43) Pub. Date: Jul. 3, 2025

(54) METHOD FOR PREPARING BIOLOGICAL VALVE MATERIAL BY COPOLYMERIZATION AND CROSSLINKING, BIOLOGICAL VALVE MATERIAL AND USE

(71) Applicant: SICHUAN UNIVERSITY, Chengdu (CN)

(72) Inventors: Yunbing Wang, Chengdu (CN); Cheng Zheng, Chengdu (CN); Li Yang, Chengdu (CN); Gaocan Li, Chengdu (CN); Rifang Luo, Chengdu (CN); Dajun Kuang, Chengdu (CN); Caili Ma, Chengdu (CN)

(21) Appl. No.: 19/084,711

(22) Filed: Mar. 19, 2025

Related U.S. Application Data
(63) Continuation-in-part of application No. PCT/CN2022/132876, filed on Nov. 18, 2022.

(30) Foreign Application Priority Data
Nov. 15, 2022 (CN) 202211431077.5

(51) Int. Cl. A61L 27/36; A61F 2/24; A61F 2/844

(52) U.S. Cl. CPC A61L 27/3687; A61F 2/2415; A61F 2/2418; A61F 2/2427; A61L 27/3625; A61F 2/844; A61F 2240/001; A61L 2430/20

(57) ABSTRACT

Disclosed is a method for preparing a biological valve material by copolymerization and crosslinking, as well as a biological valve material and use. The preparation method includes: step S110, contacting a biomaterial with an aldehyde group crosslinking agent solution for crosslinking; step S120, soaking the biomaterial treated in step S110 in a solution containing a first functional monomer for a chemical reaction to introduce a first carbon-carbon double bond, wherein the first functional monomer has the first carbon-carbon double bond and an ethylene oxide group; step S130, soaking the biomaterial treated in step S120 in a solution containing a second functional monomer, wherein the second functional monomer has a second carbon-carbon double bond and a functional group B; and step S200, performing polymerization of carbon-carbon double bonds under an action of an initiator. The method introduces carbon-carbon double bonds twice while also incorporating additional functional groups.

### CROSS-REFERENCE TO RELATED APPLICATIONS

The present disclosure is a Continuation-In-Part Application of PCT Application No. PCT/CN2022/132876, filed on Nov. 18, 2022, which claims the priority of Chinese Patent disclosure No. 202211431077.5, filed on Nov. 15, 2022, the entire contents of which are hereby incorporated by reference.

### TECHNICAL FIELD

The present application relates to the technical field of interventional materials, and in particular to a method for preparing a biological valve material by copolymerization and crosslinking, and the biological valve material and use.

### BACKGROUND

Heart valve disease is a common valvular degeneration disease. Clinically, it manifests as regurgitation caused by narrowing of the valve opening or valvular insufficiency, which poses a serious threat to the patient's life.

Prosthetic Heart Valve Replacement is the gold standard for the treatment of heart valve disease. It restores the normal opening and closing function of the valve by replacing the diseased heart valve in the patient's body with a heart valve prosthesis, which is categorized into biological valves and mechanical valves. Mechanical valves are made from synthetic materials and implanted into the patient's body through surgical thoracotomy. Biological valves, which are typically made from glutaraldehyde crosslinked animal tissue (such as porcine or bovine pericardium), exhibit excellent hydrodynamic performance with lower thrombogenicity compared to mechanical valves, so that patients do not need lifelong anticoagulation treatment after implantation. Moreover, biological valves can be implanted through minimally invasive transcatheter procedures to replace diseased heart valves, which reduces the surgical risks of valve replacement to a certain extent. Therefore, biological valves are chosen by an increasing number of patients and are gradually becoming the preferred heart valve prostheses in prosthetic heart valve replacement.

Currently, most of commercially available biological valve products are made of glutaraldehyde crosslinked porcine or bovine pericardium. Glutaraldehyde can improve the mechanical strength of the pericardium and reduce the immunogenicity of exogenous pericardium to a certain extent by crosslinking the collagen matrix of the pericardium. However, the stability and crosslinking degree of glutaraldehyde crosslinked biological valves are still not high enough. This leads to structural degradation and damage after implantation, which further compromises the structural integrity and causes structural degeneration and failure of the biological valves. Moreover, the low crosslinking degree and low stability cause the degradation of biological valve components, inducing mechanical damage and promoting calcification and deterioration, thereby impairing the normal function of the biological valve and reducing their service life. Therefore, there is an urgent need to further improve the stability and crosslinking degree of biological valves. Although biological valves have lower thrombogenicity compared to mechanical valves, they can still cause thrombosis, which may disrupt the normal function of the biological valves and bring the risk of secondary valve replacement. Additionally, the occurrence of calcification will directly lead to the deterioration of the biological valve. Therefore, the crosslinking degree, stability, anti-thrombotic and anti-calcification properties of biological valves still need to be improved.

Biological valves crosslinked by glutaraldehyde remain the most commonly used in clinical practice. In view of the fact that glutaraldehyde crosslinked biological heart valves still have problems such as stability, low degree of crosslinking, thrombosis and calcification, as well as the risks of structural degradation and failure associated with these problems, further modification of glutaraldehyde crosslinked biological heart valves not only aligns with the actual needs of actual production but also holds significant scientific value.

The applicant of the present disclosure has long been committed to the research of biological heart valves. For example, in the previous research, the Chinese patent publication No. CN 114748694A disclosed a co-crosslinked biological valve material, preparation method therefor, and use thereof, in which the biological valve material was functionally modified by introducing functional monomers for co-crosslinking at the same time of crosslinking treatment. In the biological valve preparation methods disclosed in Chinese patent publication Nos. CN 114748693A, CN 114748697A, CN 114748696A and CN 114748695A, the functional monomer was added to the functional monomer and the carbon-carbon double bonds were introduced into the functional monomer as the basis for further crosslinking, and the modification of biological valve material was completed through two rounds of crosslinking.

In the aforementioned research, whether it is introducing functional monomers for co-crosslinking modification through glutaraldehyde crosslinking, or introducing carbon-carbon double bonds as the basis for further crosslinking during the co-crosslinking process, new modified substances are introduced during the glutaraldehyde crosslinking process to participate in the crosslinking reaction.

### SUMMARY

The present disclosure provides a method for preparing a biological valve material by copolymerization and crosslinking, as well as a biological valve material and use therefor. After glutaraldehyde crosslinking, carbon-carbon double bonds are introduced stepwise to provide a controllable crosslinking chance and range for the glutaraldehyde crosslinked valve without changing the conventional glutaraldehyde crosslinking process. Meanwhile, functional groups are introduced through functional monomers to further improve the various properties of the biological valve materials.

A method for preparing a biological valve material by copolymerization and crosslinking, including:
Step S110: contacting a biomaterial with an aldehyde group crosslinking agent solution for crosslinking;
Step S120: soaking the biomaterial treated in step S110 in a solution containing a first functional monomer for a chemical reaction to introduce a first carbon-carbon double bond; wherein the first functional monomer has a first carbon-carbon double bond and an ethylene oxide group;
Step S130: soaking the biomaterial treated in step S120 in a solution containing a second functional monomer for physical permeation to introduce a second carbon-carbon double bond, wherein the second functional monomer has a second carbon-carbon double bond and a functional group B; and
Step S200, performing polymerization of carbon-carbon double bonds under an action of an initiator to obtain the biological valve material.

Optionally, the aldehyde group crosslinking agent is glutaraldehyde or formaldehyde.

Optionally, the biomaterial is animal tissue selected from one or more of the following: pericardium, valve, intestinal valve, meninges, lung valve, blood vessel, skin or ligament.

Optionally, the animal tissue is a fresh animal tissue or a decellularized biological tissue.

The step S200 includes:

Optionally, adding the initiator to a system treated in a previous step; or taking the biomaterial treated in the previous step out and directly or after washing soaking the biomaterial treated in the previous step in a solution containing the initiator.

Optionally, the initiator is a single initiator or a mixed initiator.

Optionally, the mixed initiator is:
a mixture of ammonium persulfate and sodium bisulfite, or a mixture of ammonium persulfate and sodium sulfite, or a mixture of sodium persulfate and sodium sulfite, or a mixture of potassium persulfate and sodium sulfite, or a mixture of sodium persulfate and sodium bisulfite, or a mixture of potassium persulfate and sodium bisulfite, or a mixture of potassium persulfate and tetramethylethylenediamine, or a mixture of ammonium persulfate and a mixture of tetramethylethylenediamine, or a mixture of sodium persulfate and tetramethylethylenediamine; and the concentration of each component in the mixture is in the range of 1 mM to 100 mM.

Optionally, the single initiator is any one component of each mixed initiator.

Optionally, the double-bond polymerization time is in the range of 3 h to 24 h.

Optionally, the first functional monomer is selected from at least one of the following: allyl glycidyl ether, glycidyl methacrylate and glycidyl acrylate.

Optionally, the second functional monomer is selected from one or more of the following: polyethylene glycol diacrylate, 1,4-butanediol diacrylate, ethane-1,2-diyl diacrylate, ethyl acrylate, N-methyl-2-acrylamide, N-2,2-propenyl-2-acrylamide, N-ethylacrylamide, N,N′-vinylbisacrylamide, (ethane-1,2-diylbis(oxy))bis(ethane-2,1-diyl) diacrylate, N,N′-dimethylacrylamide, N,N-dimethylmethacrylamide, and double-bond grafted polylysine.

Optionally, the functional group B is selected from at least one of the following: hydroxyl group, carboxyl group, choline carboxylate, choline sulfonate, choline phosphate, pyrrolidone, sulfonate group, carboxylate ion, sulfonate ester, sulfoxide, amide group, and methoxy group.

When the second functional monomer contains the functional group B, optionally, the second functional monomer may be selected from one of more of the following: acrylamide, acrylic acid, sodium acrylate, methacrylic acid, sodium methacrylate, 2-(prop-2-enoylamino) acetic acid, 2-acrylamido-2-methylpropanesulfonic acid, hydroxyethyl methacrylate, 3-[[2-(methacryloyloxy) ethyl]dimethylammonium] propionate, N-methyl-2-acrylamide, N-isopropylacrylamide, N-(hydroxymethyl) acrylamide, N-(2-hydroxyethyl) methacrylamide, 2-(Methacryloyloxy) ethyl]dimethyl-(3-sulfopropyl) ammonium hydroxide, 2-methacryloyloxyethylphosphorylcholine, N-(2-hydroxyethyl) acrylamide, N-(methoxymethyl) methacrylamide, 2-acrylamido-2-methylpropanesulfonic acid, and double-bond grafted hyaluronic acid.

In step S110:

Optionally, the w/w concentration of the aldehyde group crosslinking agent solution is in a range of 0.1% to 5%; and the crosslinking time is in a range of 0.5 h to 120 h.

In step S120:
the w/w concentration of the first functional monomer in the solution containing
the first functional monomer is in a range of 1% to 10%; and the reaction time is in a range of 2 h to 120 h.

Optionally, the solution containing the first functional monomer only contains the first functional monomer and a solvent that does not participate in the chemical reaction.

Optionally, the solvent in the solution containing the first functional monomer is one or more of the following: water, physiological saline, and a neutral pH buffer, and an aqueous solution of any one of methanol, ethanol, ethylene glycol, propanol, 1,2-propylene glycol, 1,3-propylene glycol, isopropanol, butanol, isobutanol, 1,2-butylene glycol, 1,3-butylene glycol, 1,4-butylene glycol, and glycerol.

In step S130:

Optionally, the v/v concentration of the second functional monomer in the solution containing the second functional monomer is in a range of 0.1% to 20%; and the immersion time is in a range of 0.5 h to 120 h.

Furthermore, the v/v concentration of the second functional monomer in the solution containing the second functional monomer is in a range of 0.1% to 6%.

Optionally, the second functional monomer permeates the biomaterial by physical penetration.

The physical penetration may be understood as when the biomaterial treated in step S120 is soaked in a solution containing the second functional monomer, the second functional monomer in the solution adheres to the surface of the biomaterial or embeds into the gaps in the biomaterial. During this process, no chemical reaction occurs between the second functional monomer and the biomaterial.

Optionally, the solution containing the second functional monomer only contains the second functional monomer and a solvent that does not participate in the reaction.

Optionally, the solvent in the solution containing the second functional monomer is one or a mixture of the following: water, physiological saline, ethanol, isopropanol or a neutral pH buffer solution.

The present disclosure further provides a biological valve material prepared by the method described.

The present disclosure further provides a method for a biological valve material, including:
Step S110: contacting the biomaterial with an aldehyde group crosslinking agent solution for crosslinking;
Step S120: soaking the biomaterial treated in step S110 in a solution containing a first functional monomer for a chemical reaction to introduce a first carbon-carbon double bond; wherein the first functional monomer has a first carbon-carbon double bond and an ethylene oxide group;
Step S130: soaking the biomaterial treated in step S120 in a solution containing a second functional monomer, wherein the second functional monomer has a second carbon-carbon double bond and a functional group B; and
Step S200, performing polymerization of carbon-carbon double bonds under an action of an initiator to obtain the biological valve material.

The present disclosure further provides a biological valve, including a stent and leaflets, wherein the leaflets are made of the biological valve material.

Optionally, the biological valve is a prosthetic heart valve.

The present disclosure further provides an interventional system, including a prosthetic heart valve and a catheter assembly, wherein the prosthetic heart valve is delivered by the catheter assembly after being folded. The prosthetic heart valve includes a stent and leaflets, and the leaflets are made of the biological valve material.

Compared with the prior art, this disclosure has at least the following benefits:
(1) In the method of this disclosure, the glutaraldehyde crosslinked biological valve material is modified by reacting with a double-bond reagent to introduce double bonds onto it. The resulting double-bond glutaraldehyde crosslinked biological valve material serves as a platform for functionalized copolymerization and crosslinking. Moreover, by initiating the polymerization between the double bonds on the glutaraldehyde-crosslinked biological valve material and the double bonds of the functional monomers, polymer of the functional monomers is introduced to form a polymer crosslinked network for functionalized copolymerization and crosslinking. This process further increases the crosslinking degree of the biological valve material and introduces functional groups. By increasing the crosslinking degree, the stability of the biological valve material is improved.
(2) In this disclosure, after introducing double bonds onto the glutaraldehyde crosslinked biological valve material, polymerization is further initiated between the carbon-carbon double bonds on the double-bond glutaraldehyde crosslinked biological valve material and the carbon-carbon double bonds of the functional monomer to introduce a functional polymer crosslinked network, which serves as a polymeric barrier to reduce the contact and interaction between collagenase in the body and the collagen matrix of the biological valve material to a certain extent. This significantly decreases the degradation of the collagen matrix on the biological valve material by collagenase, improving the stability of glutaraldehyde crosslinked biological valve material, and further reducing the risk of structural degradation of biological valve material caused by structural degeneration.
(3) In this disclosure, after introducing double bonds onto the glutaraldehyde crosslinked biological valve material, polymerization is further initiated between the carbon-carbon double bonds on the double-bond glutaraldehyde crosslinked biological valve material and the carbon-carbon double bonds of the functional monomer to introduce a functional polymer crosslinked network, which serves as a polymeric barrier to further reduce the binding of calcium ions in the environment to the mineralized zone on the biological valve material that is prone to binding with calcium ions, reducing the risk of calcification and thus playing an anti-calcification role.
(4) In this disclosure, after introducing double bonds onto the glutaraldehyde crosslinked biological valve material, polymerization is further initiated between the carbon-carbon double bonds on the double-bond glutaraldehyde crosslinked biological valve material and the carbon-carbon double bonds of the functional monomer to introduce a functional polymer crosslinked network, thereby increasing the crosslinking degree of the biological valve material and thus making the structure of the biological valve material more rigid while increasing the elasticity. Moreover, the functional polymer crosslinked network fills the gaps between the collagen matrices on the biological valve material to inhibit the deformation of collagen fibers, making the biological valve material harder while increasing the elasticity.
(5) Compared with the applicant's previous research on methods for modifying biological valve materials that introduced the carbon-carbon double bonds by adding functional monomers for co-crosslinking during glutaraldehyde modification, in the process of modifying the biological valve materials of the present disclosure, the glutaraldehyde crosslinking treatment is firstly performed and then the residual amino groups and active groups such as hydroxyl and carboxyl groups on the glutaraldehyde crosslinked valve are chemically conjugated to the functional monomers having carbon-carbon double bonds. The functional monomers with carbon-carbon double bonds undergo chemical reactions with the amino groups, hydroxyl groups and carboxyl groups on the surface of glutaraldehyde crosslinked valve through the ethylene oxide group, and the carbon-carbon double bonds are mainly grafted on the surface of the biological valve material. As there is no other substance that can participate in the crosslinking reaction during the process of glutaraldehyde crosslinking modification of the biological valve material, the original fiber structure of the biomaterial can be better protected. This can effectively ensure the mechanical properties of the valve while maintaining the orientation direction of the original fibers of the biomaterial, thereby avoiding the potential disruption of the original fiber orientation and increased fiber disorder caused by the direct addition of double-bond functional monomers during crosslinking in previous research.
(6) On the basis of chemically grafting the first carbon-carbon double bond, the second carbon-carbon double bond is further introduced through physical penetration, providing more double bonds as a crosslinking basis for secondary crosslinking, which can further increase the crosslinking degree of the biological valve material and improve the mechanical properties of the biological valve material.
(7) In this disclosure, after introducing double bonds onto the glutaraldehyde crosslinked biological valve material, polymerization is further initiated between the carbon-carbon double bonds on the double-bond glutaraldehyde crosslinked biological valve material and the carbon-carbon double bonds of the functional monomer to introduce a functional polymer crosslinked network. Since the functional polymer crosslinked network contains functional groups, the introduction of the functional polymer crosslinked network achieves both re-crosslinking and functionalization of the biological valve material. The crosslinked biological valve material after functionalized copolymerization exhibits properties corresponding to the functional groups due to their presence. The functional groups include hydroxyl group, carboxyl group, choline carboxylate, choline sulfonate, choline phosphate, pyrrolidone, sulfonate group, carboxylate ion, sulfonate ester, sulfoxide, amide group, and methoxy group, which can interact with water molecules through hydrogen bonding and ionic hydration, thereby enhancing the hydrophilicity of the biological valve material surface. This allows the formation of a hydration layer on the biological valve, which resists excessive adhesion of proteins and cells in the body, thereby improving antithrombotic properties and biocompatibility.

### BRIEF DESCRIPTION OF DRAWINGS

FIG. 1 is a process flow chart for an implementation method of post-double bond functionalized copolymerization and crosslinking in the present disclosure;

FIG. 2 is a reaction principle diagram of an embodiment method of post-double bond functionalized copolymerization and crosslinking in the present disclosure;

FIG. 3 is a scanning electron micrograph image of blood adhesion of Control Group 1 (glutaraldehyde crosslinked porcine pericardium);

FIG. 4 is a scanning electron micrograph image of blood adhesion of sample 1 in Example 1;

FIG. 5 is a scanning electron micrograph image of blood adhesion of sample 2 in Example 2;

FIG. 6 is a scanning electron micrograph image of blood adhesion of sample 7 in Example 7;

FIG. 7 is an image showing an alizarin red staining result of Control Group 1 (glutaraldehyde crosslinked porcine pericardium) after subcutaneous implantation in rats for 30 days;

FIG. 8 is an image showing an alizarin red staining result of sample 1 of Example 1 after subcutaneous implantation in a rat for 30 days;

FIG. 9 is an image showing an alizarin red staining result of sample 2 of Example 2 after being implanted subcutaneously in a rat for 30 days;

FIG. 10 is an image showing an alizarin red staining result of sample 8 of Example 8 after being implanted subcutaneously in a rat for 30 days;

FIG. 11 is a schematic structural diagram of a prosthetic heart valve of the present disclosure;

FIG. 12 is a schematic structural diagram of an interventional system of the present disclosure.

### DESCRIPTION OF EMBODIMENTS

The technical solutions according to the embodiments of the present disclosure will be described clearly and fully in combination with the drawings according to the embodiments of the present disclosure. Apparently, the described embodiments are not all embodiments of the present disclosure, but only part of the embodiments of the present disclosure. Based on the disclosed embodiments, all other embodiments obtained by those skilled in the art without creative work fall into the scope of this invention.

Unless otherwise defined, all technical and scientific terms used herein have the same meaning as commonly understood by a person skilled in the art. The terms in the description of the present disclosure are used to describe specific embodiments, and not to limit the present disclosure.

In order to improve the function of conventional glutaraldehyde crosslinked valves, the present disclosure introduces carbon-carbon double bonds on the basis of glutaraldehyde crosslinking, and further initiates a secondary crosslinking of these double bonds. This improves the anti-coagulation, anti-calcification, elasticity and other properties of glutaraldehyde-crosslinked biological heart valves. Specifically, a method for preparing a biological valve material is provided, including:
Step S100, using the amino groups on the biomaterial to introduce a first carbon-carbon double bond by chemically bonding with a first functional monomer (i.e., a double-bond reagent), wherein at least an aldehyde group crosslinking agent is present during the reaction of Step S100; and
Step S200, polymerizing the carbon-carbon double bonds under the action of an initiator to obtain the biological valve material.

The first carbon-carbon double bond introduced by chemical bonding undergoes a polymerization reaction under the action of the initiator to further form a crosslinked network, thereby improving the anti-coagulation, anti-calcification, elasticity and other properties of the glutaraldehyde crosslinked biological valve.

In step S100, it is required that the first functional monomer participates in the chemical bonding reaction. The first functional monomer further contains an ethylene oxide group as an active group, which participates in the chemical bonding.

In the present disclosure, the use of the amino groups on the biomaterial is understood to mean that at least a part of the amino groups on the biomaterial participates in the chemical reaction of introducing the first carbon-carbon double bond. In actual operation, step S100 may include multiple sub-steps. The raw materials involved in the reaction system of step S100 participate in at least one of the sub-steps, without strictly limiting the participation in the reactions of all the sub-steps.

Currently, almost all existing biological valve products used in clinical implantation are made of glutaraldehyde crosslinked biological valve materials. Glutaraldehyde reacts with collagen in the biological valve material, crosslinking the collagen matrix and reducing the immunogenicity of the material while enhancing its mechanical strength. However, the biological valve materials often exhibit low crosslinking degrees after glutaraldehyde crosslinking, making them susceptible to structural degradation. This leads to component degradation and compromises structural integrity, ultimately resulting in structural degeneration and failure. Additionally, degradation of biological valve components may in turn cause mechanical damage to the valve leaflets and promote calcification, impeding the normal opening and closing movement of the valve and reducing the lifespan of the biological valve along with the structural degeneration. Although biological valves have lower thrombogenicity compared to mechanical valves, they can still cause thrombosis, which may disrupt the normal function of the biological valves and bring the risk of secondary valve replacement. Additionally, the occurrence of calcification will directly lead to the deterioration of the biological valve.

Therefore, the crosslinking degree, stability, anti-thrombotic and anti-calcification properties of biological valves still need to be improved. Biological valves crosslinked by glutaraldehyde remain the most commonly used in clinical practice. In view of the fact that glutaraldehyde crosslinked biological heart valves still have problems such as stability, low degree of crosslinking, thrombosis and calcification, as well as the risks of structural degradation and failure associated with these problems, further modification of glutaraldehyde crosslinked biological heart valves not only aligns with the actual needs of actual production but also holds significant scientific value.

Based on the glutaraldehyde crosslinked biological valve material, the present disclosure introduces carbon-carbon double bonds on the biological heart valve material as a platform for functionalized copolymerization and crosslinking by double-bond treatment of the glutaraldehyde crosslinked biological valve material. By initiating a copolymerization reaction between the double bonds in the double-bond glutaraldehyde crosslinked biological valve material and the double bonds of the functional monomers, a functional polymer crosslinked network is introduced to the glutaraldehyde crosslinked biological valve material, thereby further expanding the crosslinked network and achieving post-double bond functionalized copolymerization and crosslinking of the biological valve material. That is, on the basis of scheme two, the second functional monomer further contains a functional group B. This will increase the crosslinking degree of the glutaraldehyde crosslinked biological valve material and improve its structural stability. The introduction of the functional polymer crosslinked network functionalizes the biological valve material, which will further improve its anti-calcification performance, anti-thrombotic performance and biocompatibility. Moreover, the introduction of the functional polymer crosslinked network enhances the crosslinking degree of the biological valve material and fills the gaps between the collagen matrix to inhibit the deformation of the collagen fibers. This further results in a relatively stiffer texture of the biological valve material while improving its elasticity.

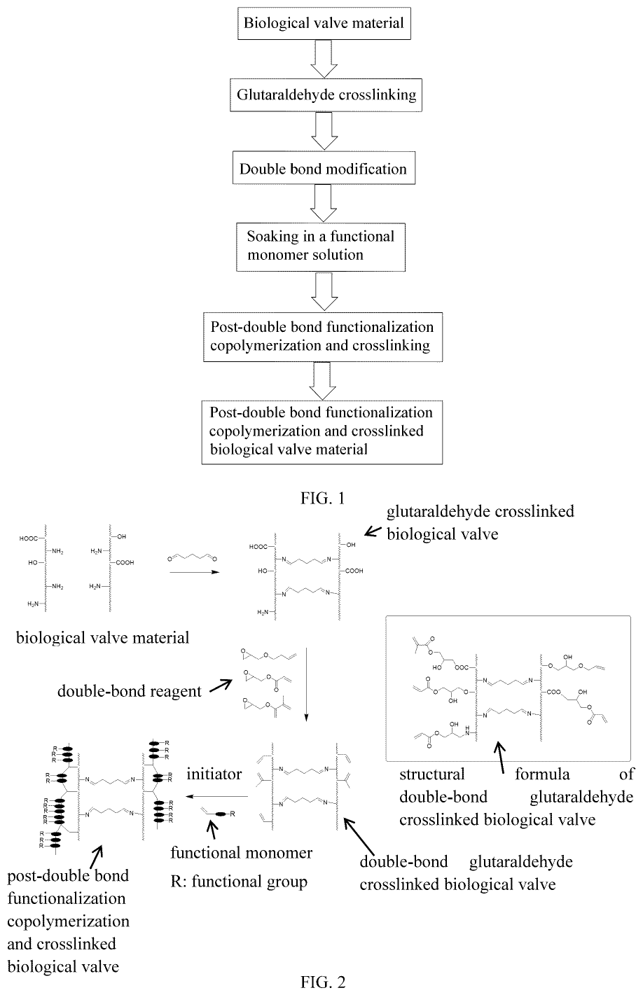

Specifically, it includes (see FIG. 1):
S110: soaking the biological valve material in an aldehyde group crosslinking agent solution for crosslinking to prepare the glutaraldehyde crosslinked biological valve material;
S120: soaking the glutaraldehyde crosslinked biological valve material prepared in step S110 in a solution of a double-bond reagent (the first functional monomer) for double-bond modification to prepare a double-bond biological valve material, wherein the double-bond reagent (the first functional monomer) has at least one first carbon-carbon double bond and an ethylene oxide group;
S130: soaking the double-bond biological valve material obtained in step S120 with a second functional monomer solution, wherein the second functional monomer has at least one second carbon-carbon double bond and at least one functional group B; and
S200: adding an initiator to the solution after the soaking in step S130 to bring the biological valve material into contact with the functional monomer solution to initiate the double-bond polymerization.

In the present disclosure, the biomaterial is first subjected to a crosslinking reaction with the aldehyde group crosslinking agent (S110), then reacts with the active group of the first functional monomer to introduce the first carbon-carbon double bond (S120), and afterward, the second carbon-carbon double bond is introduced by physical penetration of the second functional monomer (S130). During the preparation process, the aldehyde group crosslinking agent is added first, reacting with some of the amino groups on the biological valve material. Then, the first functional monomer is added, and the remaining amino groups and other groups (such as hydroxyl and carboxyl groups) on the biomaterial are used to react with the active groups of the first functional group to directly introduce the first carbon-carbon double bond. In this scheme, the active group of the first functional monomer is an ethylene oxide group. In addition to the remaining amino groups participating in the reaction, the hydroxyl and carboxyl groups of the biomaterial can also react with the ethylene oxide group to participate in the chemical reaction. On the basis of introducing the first carbon-carbon double bond by chemical reaction, the second carbon-carbon double bond is then introduced through physical penetration of the second functional monomer, which simultaneously introduces a functional group B along with the second carbon-carbon double bond. Finally, the first carbon-carbon double bond introduced by chemical reaction is further polymerized under the action of an initiator.

The schematic diagram of this disclosure is explained as follows:

In this double-bond crosslinking scheme, after the biological valve material is crosslinked with glutaraldehyde, the first carbon-carbon double bond is further introduced by using a double-bond reagent (first functional monomer) solution for double-bonding the glutaraldehyde crosslinked biological valve material, which then serves as a platform for secondary crosslinking. The double-bond reagent (first functional monomer) used has both the carbon-carbon double bond and the ethylene oxide group, and the second functional monomer has both the second carbon-carbon double bond and the functional group B.

To facilitate understanding of the chemical principles involved in this scheme, FIG. 2 is illustrated as an example for explanation. The double-bond reagent (i.e., the first functional monomer) is used to modify the glutaraldehyde crosslinked biological valve material, and a ring-opening reaction occurs between the ethylene oxide group of the double-bond reagent (i.e., the first functional monomer) and the hydroxyl groups, carboxyl groups as well as the remaining small amount of amino groups on the glutaraldehyde crosslinked biological valve material, thereby introducing the first carbon-carbon double bond onto the glutaraldehyde crosslinked biological valve material. Further, the second carbon-carbon double bond and the functional group B are introduced through the physical penetration of the second functional monomer. Still further, by initiating the polymerization of the double bonds on the glutaraldehyde crosslinked biological valve material and the double bonds of the functional monomers, the functional polymer crosslinked network is introduced to achieve further secondary crosslinking, thereby completing the post-double bond functionalized copolymerization and crosslinking of the biological valve material.

Since there are more functional groups (hydroxyl and carboxyl groups other than amino groups) on the biological valve material used for crosslinking, the functional polymer crosslinked network is further introduced through copolymerization with functional monomers, and the biological valve material has a larger crosslinked network. The crosslinking degree of the biological valve material treated with post-double bond functionalized copolymerization and crosslinking will be significantly improved, and its structural stability and anti-calcification performance will also be significantly improved with the introduction of the functional polymer crosslinked network.

The carbon-carbon double bonds are introduced after crosslinking with glutaraldehyde, they are mainly grafted on the surface of the biological valve material. As there is no other substance that can participate in the crosslinking reaction added in the process of glutaraldehyde crosslinking modification of the biological valve material, the original fiber structure of the biomaterial can be better protected. This can effectively ensure the mechanical properties of the valve while maintaining the orientation direction of the original fibers of the biomaterial, thereby avoiding the potential disruption of the original fiber orientation and increased fiber disorder caused by the direct addition of double-bond functional monomers during crosslinking in previous research.

On the basis of chemically grafting the first carbon-carbon double bond, the second carbon-carbon double bond is further introduced through physical penetration, providing more double bonds as a crosslinking basis for secondary crosslinking, which can further increase the crosslinking degree of the biological valve material and improve its mechanical properties.

Moreover, the second functional monomer further contains functional groups, thereby enriching the biological valve material with these functional groups and endowing the biological valve material with properties corresponding to these functional groups. The functional group B may be selected from hydroxyl group, carboxyl group, choline carboxylate, choline sulfonate, choline phosphate, pyrrolidone, sulfonate group, carboxylate ion, sulfonate ester, sulfoxide, amide group, and methoxy group, which interact with water molecules through hydrogen bonding and ionic hydration, thereby enhancing the hydrophilicity of the biological valve material surface. This allows the formation of a hydration layer on the biological valve, which resists excessive adhesion of proteins and cells in the body, thereby improving antithrombotic properties and biocompatibility.

Regarding the introduced functional group B:
Hydroxyl group: as a hydrophilic group, it improves the surface hydrophilicity of the biomaterial to achieve anti-coagulation effect.
Carboxyl group: as a hydrophilic group, it improves the surface hydrophilicity of the biomaterial to achieve anti-coagulation effect.
Carboxylate ion and sulfonate group: they improve the surface hydrophilicity of the biomaterial through ion hydration to achieve anti-coagulation effect.
Sulfoxide and pyrrolidone: as hydrophilic groups, they improve the surface hydrophilicity of the biomaterial to achieve anticoagulant effects.
Zwitterion: It improves the surface hydrophilicity of the biomaterial through ion hydration to achieve anti-coagulation effect. This is beneficial to form an electrically neutral surface of the biological valve, thereby reducing the adsorption of calcium ions and achieving anti-calcification effect.
Polyethylene glycol: as a hydrophilic group, it improves the surface hydrophilicity of the biomaterial, as well as increases the steric hindrance between calcium ions and collagen, thereby improving the surface hydrophilicity of the biological valve material.
Carbamate group: as a hydrophilic group, it improves the surface hydrophilicity of the biomaterial to achieve an anti-coagulation effect.
Amide: as a hydrophilic group, it improves the surface hydrophilicity of the biomaterial to achieve an anti-coagulation effect; and as a toughening group, it can dynamically adjust the elasticity of the biomaterial to improve the utilization rate, resulting in superior hemodynamic performance of the prepared valve.

Optionally, in step S120 of the present disclosure, the first carbon-carbon double bond is introduced through a non-condensation chemical bonding.

Optionally, in step S110, the biomaterial does not undergo any chemical reactions involving other reagents before being treated with the aldehyde group crosslinking agent.

Further optionally, in the reaction system of step S120, the first carbon-carbon double bonds are provided by the first functional monomer having the active group, and the reaction raw materials in step S100 only include the biomaterial, the first functional monomer and the aldehyde group crosslinking agent.

In step S110:

The crosslinking agent in the present disclosure is the aldehyde group crosslinking agent used in current mainstream crosslinking methods. Optionally, the aldehyde group crosslinking agent is selected from glutaraldehyde and formaldehyde.

Optionally, the concentration of the glutaraldehyde solution is in the range of 0.1% to 5% (w/w), and the crosslinking time may be any time in the range of 0.5 h to 120 h.

The biomaterials used here are conventional biomaterials used in the existing glutaraldehyde crosslinking processes with collagen content ranging from 60% to 90%. Further, the biomaterials are animal tissues originating from the pig, cattle, horse or sheep, including one or more of the following: pericardium, valve, intestinal valve, meninges, lung valve, blood vessel, skin or ligament.

Optionally, the animal tissue is fresh animal tissue or decellularized biological tissue.

Optionally, in the decellularization, the biological tissue is treated with a surfactant as follows:
the biological tissue is decellularized with an ionic surfactant; or
the biological tissue is decellularized with a non-ionic surfactant.

The ionic surfactant is mainly used to lyse cells, and the non-ionic surfactant is mainly used to remove lipid substances (such as phospholipids).

Optionally, the ionic surfactant is at least one of the following: sodium deoxycholate, fatty acid potassium soap, sodium dodecyl sulfate, sodium cholate, hexadecyltrimethylammonium bromide, fatty acid potassium salt, and alkyldimethylsulfonpropyl betaine.

Optionally, the nonionic surfactant is at least one of Triton and Tween.

In step S120,
optionally, the double-bond reagent, i.e., the first functional monomer, is selected from at least one of allyl glycidyl ether, glycidyl methacrylate and glycidyl acrylate.

Optionally, the concentration of the double-bond reagent in the solution containing the first functional monomer (i.e., the double-bond reagent) is in the range of 1% to 10% (w/w), and the reaction time for the double-bond modification is in the range of 2 h to 120 h.

Optionally, the solvent in the solution containing the first functional monomer, i.e., the double-bond reagent, is one or more of the following: water, physiological saline, neutral pH buffer, or an aqueous solution of methanol, ethanol, ethylene glycol, propanol, 1,2-propylene glycol, 1,3-propylene glycol, isopropanol, butanol, isobutanol, 1,2-butylene glycol, 1,3-butylene glycol, 1,4-butylene glycol, and glycerol.

Optionally, the biofilm material treated in S110 is taken out and washed or directly placed in the solution containing a double-bond reagent (first functional monomer).

In step S130,
the biological valve material treated in step S120 is soaked in the second functional monomer solution directly or after washing.

Optionally, the second functional monomer has at least a second carbon-carbon double bond and at least one functional group B.

Optionally, the second functional monomer is one or more of the following: acrylamide, acrylic acid, sodium acrylate, methacrylic acid, sodium methacrylate, 2-(prop-2-enoylamino) acetic acid, 2-acrylamido-2-methylpropanesulfonic acid, hydroxyethyl methacrylate, 3-[[2-(methacryloyloxy) ethyl]dimethylammonium] propionate, N-methyl-2-acrylamide, N-isopropylacrylamide, N-(hydroxymethyl) acrylamide, N-(2-hydroxyethyl) methacrylamide, 2-(Methacryloyloxy) ethyl]dimethyl-(3-sulfopropyl) ammonium hydroxide, 2-methacryloyloxyethyl phosphorylcholine, N-(2-hydroxyethyl) acrylamide, N-(methoxymethyl) methacrylamide, 2-acrylamido-2-methylpropanesulfonic acid, and double-bond grafted hyaluronic acid (which may be prepared by the method as described above)

Optionally, the concentration of the second functional monomer solution is in the range of 0.1% to 6% (v/v).

Optionally, the solvent of the second functional monomer solution is one or a mixture of the following: water, physiological saline, ethanol, isopropanol or a neutral pH buffer solution.

Optionally, the immersion time in the second functional monomer solution is in the range of 0.5 h to 120 h.

In step S200,
the biological valve material treated in step S120 is cleaned with deionized water and then soaked in the initiator solution, or the initiator is directly added to the reaction system in step S120 to initiate the polymerization reaction; wherein the latter is commonly known as the one-pot method;

Optionally, the solvent in the solution containing the initiator is water, physiological saline or neutral pH buffer.

Regarding the concentration of the initiator as mentioned above, in the one-pot method, it may be understood as the concentration of the solution of the reaction system in step S120 in the one pot method, while in the step-by-step method, it may be understood as the concentration of the solution containing the initiator.

Optionally, the initiator is a mixture of ammonium persulfate and sodium bisulfite, or a mixture of ammonium persulfate and sodium sulfite, or a mixture of sodium persulfate and sodium sulfite, or a mixture of potassium persulfate and sodium sulfite, or a mixture of sodium persulfate and sodium bisulfite, or a mixture of potassium persulfate and sodium bisulfite, or a mixture of potassium persulfate and tetramethylethylenediamine, or a mixture of ammonium persulfate and tetramethylethylenediamine, or a mixture of sodium persulfate and tetramethylethylenediamine; and the concentration of each component in the mixture is in the range of 1 mM to 100 mM.

The reaction time of step S200 is in the range of 3 to 24 hours.

In the present disclosure, all reactions in S100 and S200 can be carried out at 0 to 50° C. unless otherwise specified. Preferably, the temperature does not need to be specially controlled and can be at room temperature. It is suitable not to exceed the human body's adaptive temperature, preferably in the range of 36° C. to 37° C.

In the present disclosure, all reactions in S100 and S200 may be either static reactions or dynamic reactions unless otherwise specified. Dynamic reactions may be conducted using equipment capable of circulating the solution, such as a peristaltic pump, or through shaking at a rotation speed ranging from 10 rpm to 150 rpm. The peristaltic cycle or shaking may be carried out continuously or intermittently.

In the present disclosure, optionally, dehydration and drying treatment after the double-bond polymerization is completed are carried out to prepare a dry valve. Further, after the double-bond polymerization is completed, the biological valve material is conventionally cleaned, softened, and then dehydrated and dried.

The cleaning solution may be one or more of water, physiological saline, ethanol, isopropanol or neutral pH buffer solution. The pH may be adjusted to be in the range of 5.0 to 9.5 before and during use, or there is no need to adjust the pH.

Optionally, the dehydration treatment involves exposing the valve piece subjected to double-bond polymerization or the valve formed by sewing valve pieces to a dehydration solution.

Optionally, the dehydration solution is a mixed solution of alcohol solution and water with the alcohol solution accounting for 20% to 90% (v/v). The alcohol reagent may be one or both of ethanol and isopropanol.

Optionally, the drying treatment involves exposing the dehydrated valve piece or valve to a softener solution for 20 minutes to 10 hours.

Optionally, the main component of the softener solution is one or a mixed solution of glycerol and polyethylene glycol, with the glycerol concentration ranging from 10% to 100% (v/v), and the remaining component is one or more of water, ethanol, isopropanol, etc., accounting for 0 to 90% (v/v).

Optionally, the valve sterilization method after drying treatment may be one of ethylene oxide sterilization or electron beam sterilization.

The biological valve material prepared by the above methods may be used for interventional biological valves, for example, through minimally invasive intervention; or the biological valve material prepared by the above methods may be used for surgical biological valves, for example, through surgical implantation.

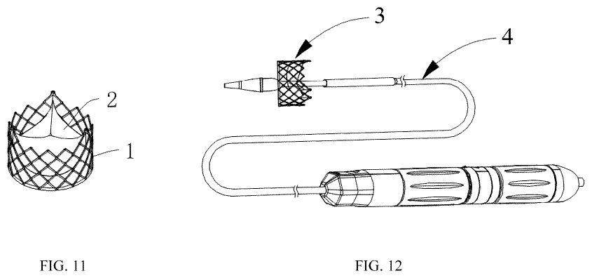

As shown in FIG. 11, a prosthetic heart valve according to an embodiment includes a stent 1 and leaflets 2 connected in the stent 1. The stent is generally cylindrical, and the side wall thereof has a hollow mesh structure. There is a blood flow channel defined in the stent, and the leaflets cooperate with each other to control the opening and closing of the blood flow channel in the stent.

Depending on the release modes, the stent is made of corresponding materials during processing, such as nickel-titanium alloy that has shape memory and can self-expand in the human body, or stainless steel that is released by ball expansion, etc. The stent itself may be formed by cutting pipes or braiding wires. The leaflets may be connected to the stent by sewing, bonding or integral molding.

For positioning in the human body, positioning structures that can interact with surrounding native tissues may be provided on the periphery of the stent, such as anchors, arms, etc. In order to prevent peripheral leakage, skirts or anti-peripheral leakage materials or the like may be provided on the inside and/or outside of the stent. The leaflets, skirts or anti-peripheral leakage materials may all be made of the biological valve materials in the above embodiments.

As shown in FIG. 12, in case of intervention through catheter, the prosthetic heart valve 3 and a corresponding delivery system form a valve intervention system. The delivery system includes a catheter assembly 4 and a handle for controlling the catheter assembly. The prosthetic heart valve is in a radially compressed state during delivery in the human body, and is radially expanded in the human body after releasing the restriction of the catheter assembly or being expanded by ball expansion.

Specific examples are illustrated in the following.

### Control Example 1

During the treatment process, a simple glutaraldehyde crosslinked group was set as a control group, and the porcine pericardium was immersed in 0.25% (w/w) glutaraldehyde at room temperature for 72 hours to prepare the glutaraldehyde crosslinked porcine pericardium, which was recorded as control sample 1.

### Example 1

Freshly collected porcine pericardium was soaked in physiological saline and washed by shaking for 2 hours, and then soaked in a 0.25% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water, and then soaked in an isopropanol aqueous solution of 5% (v/v) glycidyl methacrylate at room temperature for double-bond modification of the glutaraldehyde crosslinked porcine pericardium. The reaction time was 48 hours, and the solvent of the double-bond solution was a 20% (v/v) propanol aqueous solution.

After the double-bond modification was completed, the double-bond glutaraldehyde crosslinked porcine pericardium was washed with deionized water; and then the double-bond glutaraldehyde crosslinked porcine pericardium was soaked in a 3% (w/v) 2-(Methacryloyloxy) ethyl]dimethyl-(3-sulfopropyl) ammonium hydroxide solution for 2 hours.

An initiator which included ammonium persulfate at a concentration of 20 mM and sodium bisulfite at a concentration of 10 mM was added to the above solution to further initiate polymerization between the double bonds on the double-bond glutaraldehyde crosslinked biological valve material and the double bonds of 2-(Methacryloyloxy) ethyl]dimethyl-(3-sulfopropyl) ammonium hydroxide. After reacting at 37° C. for 8 hours, a porcine pericardium crosslinked after double-bond copolymerization was obtained, which was recorded as sample 1.

### Example 2

Freshly collected porcine pericardium was soaked in physiological saline and washed by shaking for 2 hours, and then soaked in a 0.25% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an aqueous solution of 6% (v/v) propanol glycidyl acrylate at room temperature for double-bond modification of the glutaraldehyde crosslinked porcine pericardium. The reaction time was 72 hours, and the solvent of the double-bond solution used was a 20% (v/v) propanol aqueous solution.

After the double-bond modification was completed, the double-bond glutaraldehyde crosslinked porcine pericardium was washed with deionized water; and then the double-bond glutaraldehyde crosslinked porcine pericardium was soaked in a 5% (w/v) 2-methacryloyloxyethylphosphocholine solution for 1 hour.

An initiator which included ammonium persulfate at a concentration of 20 mM and sodium bisulfite at a concentration of 10 mM was added to the above solution to further initiate polymerization between the double bonds on the double-bond glutaraldehyde crosslinked biological valve material and the double bonds of 2-methacryloyloxyethylphosphocholine. After reacting at 37° C. for 8 hours, a porcine pericardium crosslinked after double-bond copolymerization was obtained, which was recorded as sample 2.

### Example 3

Freshly collected porcine pericardium was soaked in physiological saline and washed by shaking for 2 hours, and then soaked in a 0.25% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an isopropanol aqueous solution with 2% (v/v) glycidyl acrylate and 4% (v/v) allyl glycidyl ether at room temperature for double-bond modification of the glutaraldehyde-crosslinked porcine pericardium. The reaction time was 72 hours, and the solvent of the double-bond solution was a 30% (v/v) aqueous ethanol solution.

After the double-bond modification was completed, the double-bond glutaraldehyde crosslinked porcine pericardium was washed with deionized water; and then the double-bond glutaraldehyde crosslinked porcine pericardium was soaked in a 5% (v/v) acrylamide solution for 3 hours.

An initiator which included ammonium persulfate at a concentration of 20 mM and sodium bisulfite at a concentration of 10 mM was added to the above solution to further initiate the polymerization between the double bonds on the double-bond glutaraldehyde crosslinked biological valve material and the double bonds of acrylamide. After reacting at 37° C. for 8 hours, a porcine pericardium crosslinked after double-bond copolymerization was obtained, which was recorded as sample 3.

### Example 4

Freshly collected porcine pericardium was soaked in physiological saline and washed by shaking for 2 hours, and then soaked in a 1.0% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an isopropanol aqueous solution with 3% (v/v) glycidyl methacrylate and 2% (v/v) glycidyl acrylate at room temperature for double-bond modification of the glutaraldehyde crosslinked porcine pericardium. The reaction time was 48 hours, and the solvent of the double-bond solution was a 25% (v/v) isopropanol aqueous solution.

After the double-bond modification was completed, the double-bond glutaraldehyde crosslinked porcine pericardium was washed with deionized water; and then the double-bond glutaraldehyde crosslinked porcine pericardium was soaked in a solution containing 1% (v/v) acrylamide and 1.5% (v/v) N-isopropylacrylamide for 1 hour.

An initiator which included ammonium persulfate at the concentration of 20 mM and sodium bisulfite at the concentration of 10 mM was added to the above solution to further initiate the polymerization between the double bonds on the double-bond glutaraldehyde crosslinked biological valve material and the double bonds of acrylamide and N-isopropylacrylamide. After reacting at 37° C. for 7 hours, a porcine pericardium crosslinked after double-bond copolymerization was obtained, which was recorded as sample 4.

### Example 5

Freshly collected porcine pericardium was soaked in physiological saline and washed by shaking for 2 hours, and then soaked in a 0.25% (w/w) glutaraldehyde solution at room temperature and shaken for 72 hours to perform glutaraldehyde group crosslinking treatment on the porcine pericardium to prepare glutaraldehyde crosslinked porcine pericardium.

The glutaraldehyde crosslinked porcine pericardium was washed with deionized water and soaked in an ethanol aqueous solution of 4% (v/v) glycidyl methacrylate at room temperature for double-bond modification. The reaction time was 72 hours and the solvent of the double-bond solution was a 20% (v/v) ethanol aqueous solution.

After the double-bond modification was completed, the double-bond glutaraldehyde crosslinked porcine pericardium was washed with deionized water; and then the double-bond glutaraldehyde crosslinked porcine pericardium was soaked in a 1.5% (v/v) N-isopropylacrylamide solution for 1 hour.

An initiator which included sodium persulfate at the concentration of 20 mM and sodium bisulfite at the concentration of 7 mM was added to the above solution to further initiate polymerization between the double bonds on the double-bond glutaraldehyde crosslinked biological valve material and the double bonds of N-isopropylacrylamide. After reacting at 37° C. for 7 hours, a porcine pericardium crosslinked after double-bond copolymerization was obtained, which was recorded as sample 5.

### Example 6

Freshly collected porcine pericardium was soaked in physiological saline and washed by shaking for 2 hours, and then soaked in a 0.25% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an isobutanol aqueous solution of 4% (v/v) glycidyl methacrylate at room temperature for double-bond modification of the glutaraldehyde crosslinked porcine pericardium. The reaction time was 72 hours, and the solvent of the double-bond solution was a 15% (v/v) isobutanol aqueous solution.

After the double-bond modification was completed, the double-bond glutaraldehyde crosslinked porcine pericardium was washed with deionized water; and then the double-bond glutaraldehyde crosslinked porcine pericardium was soaked in a 2.0% (w/v) sodium acrylate solution for 5 hours.

An initiator which included ammonium persulfate at a concentration of 20 mM and sodium bisulfite at a concentration of 10 mM was added to the above solution to further initiate the polymerization between the double bonds on the double-bond glutaraldehyde crosslinked biological valve material and the double bonds of sodium acrylate. After reacting at 37° C. for 12 hours, a porcine pericardium crosslinked after double-bond copolymerization was obtained, which was recorded as sample 6.

### Example 7

Freshly collected porcine pericardium was soaked in physiological saline and washed by shaking for 2 hours, and then soaked in a 0.25% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an isopropanol aqueous solution of 4% (v/v) glycidyl acrylate at room temperature for double-bond modification. The reaction time was 48 hours and the solvent of the double-bond solution was 20% (v/v) methanol aqueous solution.

After the double-bond modification was completed, the double-bond glutaraldehyde crosslinked porcine pericardium was washed with deionized water; and then the double-bond glutaraldehyde crosslinked porcine pericardium was soaked in a solution containing 1.0% (v/v) hydroxyethyl methacrylate and 0.5% (w/v) 2-(Methacryloyloxy) ethyl]dimethyl-(3-sulfopropyl) ammonium hydroxide for 1 hour.

An initiator which included ammonium persulfate at a concentration of 20 mM and sodium bisulfite at a concentration of 10 mM was added to the above solution to further initiate the polymerization between the double bonds on the double-bond glutaraldehyde crosslinked biological valve material and the double bonds of hydroxyethyl methacrylate and 2-(Methacryloyloxy)ethyl]dimethyl-(3-sulfopropyl) ammonium hydroxide. After reacting at 37° C. for 8 hours, a porcine pericardium crosslinked after double-bond copolymerization was obtained, which was recorded as sample 7.

### Example 8

Freshly collected porcine pericardium was soaked in physiological saline and washed by shaking for 2 hours, and then soaked in a 0.25% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an ethylene glycol aqueous solution of 5% (v/v) glycidyl methacrylate at room temperature for double-bond modification of the glutaraldehyde crosslinked porcine pericardium. The reaction time was 72 hours, and the solvent of the double-bond solution was a 25% (v/v) ethylene glycol aqueous solution.

After the double-bond modification was completed, the double-bond glutaraldehyde crosslinked porcine pericardium was washed with deionized water; and then the double-bond glutaraldehyde crosslinked porcine pericardium was soaked in a 5% (v/v) N-(hydroxymethyl) acrylamide solution for 5 hours.

An initiator which included ammonium persulfate at a concentration of 20 mM and sodium bisulfite at a concentration of 10 mM was added to the above solution to further initiate the polymerization between the double bonds on the double-bond glutaraldehyde crosslinked biological valve material and the double bonds of N-(hydroxymethyl) acrylamide. After reacting at 37° C. for 12 hours, a porcine pericardium crosslinked after double-bond copolymerization was obtained, which was recorded as sample 8.

### Example 9

Freshly collected porcine pericardium was soaked in physiological saline and washed by shaking for 2 hours, and then soaked in a 0.25% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in a propanol aqueous solution of 7% (v/v) glycidyl acrylate at room temperature for double-bond modification. The reaction time was 60 hours, and the solvent of the double-bond solution was a 30% (v/v) propanol aqueous solution.

After the double-bond modification was completed, the double-bond glutaraldehyde crosslinked porcine pericardium was washed with deionized water; and then the double-bond glutaraldehyde crosslinked porcine pericardium was soaked in a 1.0% (v/v) N-(methoxymethyl) methacrylamide solution for 5 hours.

Initiators which included ammonium persulfate at a concentration of 20 mM and tetramethylethylenediamine at a concentration of 1 mM to the above solution to further initiate polymerization between the double bonds on the double-bond glutaraldehyde crosslinked biological valve material and the double bonds of N-(methoxymethyl) methacrylamide. After reacting at 37° C. for 12 hours, a porcine pericardium crosslinked after double-bond copolymerization was obtained, which was recorded as sample 9.

### Example 10

Freshly collected porcine pericardium was soaked in physiological saline and washed by shaking for 2 hours, and then soaked in a 0.25% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was washed with deionized water and soaked in an isopropanol aqueous solution containing 4% (v/v) glycidyl methacrylate and 2% (v/v) glycidyl acrylate at room temperature for double-bond modification. The reaction time was 84 hours, and the solvent of the double-bond solution was a 25% (v/v) ethanol aqueous solution.

After the double-bond modification was completed, the double-bond glutaraldehyde crosslinked porcine pericardium was washed with deionized water; then, the double-bond glutaraldehyde crosslinked porcine pericardium was soaked in a solution containing 1% (v/v) acrylamide and 1.50% (w/v) 2-methacryloyloxyethylphosphocholine for 3 hours.

An initiator which included ammonium persulfate at a concentration of 20 mM and sodium bisulfite at a concentration of 10 mM was added to the above solution to further initiate polymerization between the double bonds on the double-bond glutaraldehyde crosslinked biological valve material and the double bonds of acrylamide and 2-methacryloyloxyethylphosphocholine. After reacting at 37° C. for 7 hours, a porcine pericardium crosslinked after double-bond copolymerization was obtained, which was recorded as sample 10.

### Performance Characterization of Samples of Examples 1 to 10 and Control Example 1

In order to characterize the change in the crosslinking degree of glutaraldehyde crosslinked biological valve materials before and after double-bond polymerization and crosslinking treatment, the thermal stability and crosslinking degree of biological valve materials were characterized by measuring the thermal shrinkage temperature of biological valve materials; the stability of biological valve materials was characterized through enzyme degradation experiment; the degree of calcification (anti-calcification performance) of the samples were characterized through rat subcutaneous implantation experiment, and the elastic angle of the biological valve material was tested to characterize its elasticity.

### Thermal Shrinkage Temperature Determination:

The biological valve material was cut into a circular sheet with a diameter of 0.6 cm, which was placed in a crucible after drying, and then the thermal shrinkage temperature of the biological valve material was measured on a differential scanning calorimeter in the range of 40° C. to 120° C. at a heating rate of 10° C./min. Thermal stability and crosslinking degree of the biological valve material were characterized by measuring the thermal shrinkage temperature. A higher heat shrinkage temperature corresponds to a higher degree of thermal stability and crosslinking.

TABLE 1
Thermal shrinkage temperature of biological valve materials

| Sample | Thermal shrinkage temperature (° C.) |
|---|---|
| Control Group 1 (glutaraldehyde | 86.7 |
| crosslinked porcine pericardium) | |
| Sample 1 | 89.4 |
| Sample 3 | 91.8 |
| Sample 6 | 90.5 |
| Sample 8 | 92.4 |

Through the thermal shrinkage temperature measurement of sample 1, sample 3, sample 6, sample 8 and the Control Group 1 (glutaraldehyde crosslinked porcine pericardium), it can be seen that, as shown in Table 1, the thermal shrinkage temperatures of sample 1, sample 3, sample 6, sample 8 are all higher than that of Control Group 1 (glutaraldehyde crosslinked porcine pericardium), that is, the thermal stability and crosslinking degree of sample 1, sample 3, sample 6, sample 8 are all higher than those in the Control Group 1 (glutaraldehyde crosslinked porcine pericardium). The experimental results of the thermal shrinkage temperature measurement show that the method for preparing biological valve materials by post-double bond functionalized copolymerization and crosslinking of the present disclosure can improve the thermal stability and crosslinking degree of biological valves.

### Water Contact Angle Test

The biological valve material was cut into sheets of 1*1 cm², frozen at 80° C. overnight, and then transferred to a freeze dryer for freeze drying for 4 to 8 hours. The sheets were taken out and placed on a water contact angle tester to measure the water contact angles of different materials to characterize the hydrophilicity of the materials. The smaller the water contact angle obtained, the more hydrophilic the biological valve material is.

TABLE 2
Water contact angle of biological valve materials

| Sample | Water contact angle (°) |
|---|---|
| Control Group 1 (glutaraldehyde | 67 |
| crosslinked porcine pericardium) | |
| Sample 1 | 26 |
| Sample 2 | 42 |
| Sample 7 | 45 |
| Sample 10 | 33 |

The water contact angle test results are shown in Table 2. Compared with the Control Group 1 (glutaraldehyde crosslinked porcine pericardium), the water contact angles of samples 1, 2, 7 and 10 are significantly decreased, that is, samples 1, 2, 7 and 10 are more hydrophilic than the Control Group 1 (glutaraldehyde crosslinked porcine pericardium), which indicates that the method of preparing biological valve materials by post-double bond functionalized copolymerization and crosslinking can improve the hydrophilicity of biological valves.

### Blood Adhesion Test

The biological valve material was cut into a circular sheet with a diameter of 1 cm and transferred to a 48-well plate. Then, 0.5 mL of fresh rabbit blood was added to the surface of the material to ensure sufficient contact with the blood for the blood adhesion experiment. After 1.5 hours of contact with blood, the biological valve material was removed from the blood and washed three times with physiological saline. The washed biological valve material was immersed in a 2.5% (w/v) glutaraldehyde solution and fixed for 2 hours. After fixation, the biological valve material was dehydrated with gradient concentrations of ethanol (50%, 75%, 90% and 100%, v/v), followed by gold sputtering. Finally, the material was placed under a scanning electron microscope for observation and imaging of blood adhesion to characterize its antithrombotic properties.

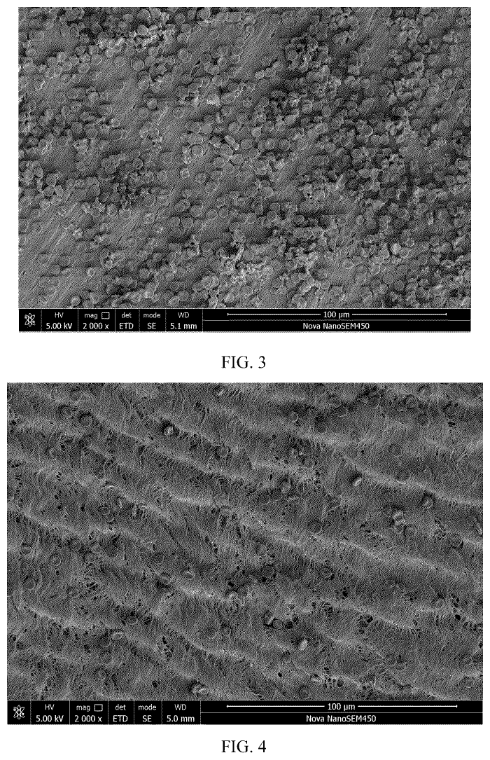

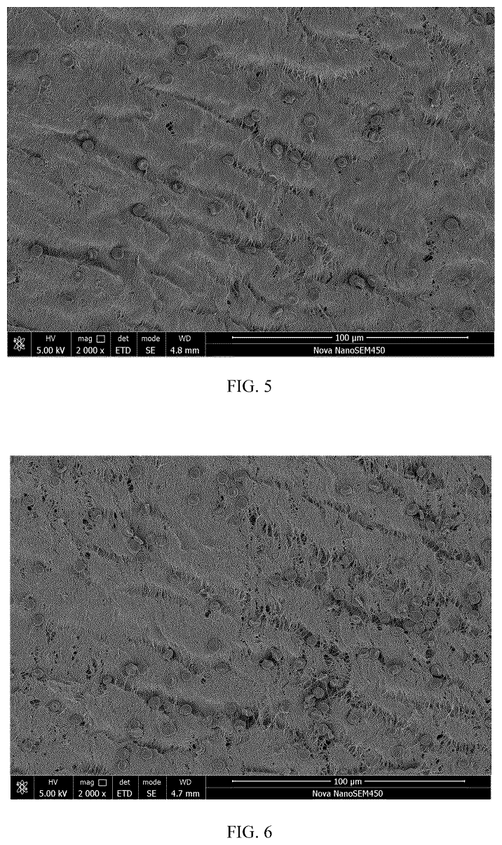

Results Analysis: as shown in FIGS. 3 to 6: After contact with blood, a large amount of red blood cell and platelet were observed to adhere to the Control Group 1 (glutaraldehyde crosslinked porcine pericardium) (FIG. 3), while only a small amount of red blood cell was observed to adhere to sample 1 (FIG. 4), sample 2 (FIG. 5), and sample 7 (FIG. 6). The blood cell adhesion on sample 1, sample 2, and sample 7 was low, reducing the interaction between blood and biological valve materials, which further reduced the possibility of thrombosis on biological valve materials, that is, sample 1, sample 2, and sample 7 had better anti-thrombotic properties than Control Group 1 (glutaraldehyde crosslinked porcine pericardium). The blood adhesion tests have shown that the method of preparing biological valve materials by post-double bond functionalized copolymerization and crosslinking can improve the anti-thrombotic properties of biological valves.

### Elasticity Test Experiment

The biological valve material with uniform thickness was cut into a rectangular sample of 1*4.6 cm², which was clamped horizontally along the midline of the long side thereof. The angle at which the sample sag relative to the horizontal plane of the midline was tested to characterize the elasticity of the sample. The smaller the angle, the higher the elasticity.

TABLE 3
Elastic angle of biological valve materials

| Sample | Elastic angle (°) |
|---|---|
| Control Group 1 (glutaraldehyde | 60 |
| crosslinked porcine pericardium) | |
| Sample 1 | 50 |
| Sample 3 | 35 |
| Sample 6 | 45 |
| Sample 8 | 30 |

The elasticity test experiments were conducted on sample 1, sample 3, sample 6, sample 8 and Control Group 1 (glutaraldehyde crosslinked porcine pericardium) to characterize their elasticity. The results of the elasticity test are shown in Table 3. Compared with Control Group 1 (glutaraldehyde crosslinked porcine pericardium), the elastic angles of sample 1, sample 3, sample 6, sample 8 were lower, indicating that their elasticity was significantly improved than the Control Group (glutaraldehyde crosslinked porcine pericardium). The method of preparing biological valve materials by post-double bond functionalized copolymerization and crosslinking can improve the elasticity of biological valve materials, which is conducive to rapid recovery of shape after transcatheter implantation.

### Enzyme Degradation Experiment

The obtained biological valve material was cut into circular sheets with a diameter of 1 cm, with each group consisting of 6 to 8 parallel test samples. All circular sheet samples were placed on a 48-well plate, frozen overnight at −80° C., and then transferred to a vacuum freeze-dryer to freeze-dry for 48 h. Each sample was weighed on a microbalance as the initial weight (W₀) and placed back into the 48-well plate. 0.5 mL collagenase I solution in PBS was added with a pipette to the wells of the 48-well plate so that the biological valve sample is completely soaked in the collagenase solution in PBS (100 U/mL). Then the 48-well plate was transferred to a 37° C. constant temperature incubator for incubation for 24 h. After the incubation, the biological valve material sample was taken out. After repeated flushing three times, the sample was frozen overnight at −80° C. and then transferred to a vacuum freeze-dryer to freeze-dry for 48 h. Each sample after degradation by collagenase solution was weighed on a microbalance as the final weight (Wₜ). The calculation formula for enzymatic degradation weight loss rate is as follows:

$$Enzyme degradation weight loss rate = \frac{{W}_{0} - {W}_{t}}{{W}_{0}} \times 100 \% .$$

The collagenase degradation weight loss rate was measured for sample 1, sample 3, sample 6, sample 8 and Control Group 1. The results are shown in Table 4.

TABLE 4
Enzyme degradation weight loss rate of biological valve materials

| | Enzyme degradation weight loss rate |
|---|---|
| Sample | (%) |
| Control Group 1 (glutaraldehyde | 6.35 ± 0.89 |
| crosslinked porcine pericardium) | |
| Sample 1 | 3.12 ± 0.30 |
| Sample 3 | 2.65 ± 0.47 |
| Sample 6 | 4.90 ± 0.45 |
| Sample 8 | 1.15 ± 0.26 |

The enzyme degradation experiments were conducted on sample 1, sample 3, sample 6, sample 8 and Control Group 1 (glutaraldehyde crosslinked porcine pericardium) to characterize the crosslinking degree of samples, and the enzymatic degradation weight loss rates of samples after sample 1, sample 2, sample 5, sample 10 and Control Group 1 were treated with collagenase I were then calculated as shown in Table 4. The enzymatic degradation weight loss rates of sample 1, sample 3, sample 6, sample 8 were all lower than that of the Control Group 1 (glutaraldehyde crosslinked porcine pericardium), which indicates that sample 1, sample 3, sample 6, sample 8 have higher stability than the Control Group (glutaraldehyde crosslinked porcine pericardium), that is, sample 1, sample 3, sample 6, sample 8 are more stable. The results of the enzyme degradation experiment show that the method for preparing biological valve materials by post-double bond functionalized copolymerization and crosslinking of the present disclosure can improve the stability of biological valve materials.

### Anti-Calcification Test

The biological valve materials were cut into 1*1 cm² sheets, sterilized and implanted subcutaneously in rats for 30 days before being taken out. Each sample was divided into two parts. One part was decapsulated and freeze-dried and weighed, and then digested with 6 M hydrochloric acid to measure the calcium content per gram of sample. The other part of the sample was fixed with paraformaldehyde tissue fixative. After fixation, the samples were taken out and smoothed with a scalpel, and then transferred to a dehydration box. The material samples were dehydrated using gradient ethanol. After dehydration, the material samples were transferred to an embedding machine and embedded in melted wax, and then transferred to a −20° C. refrigerator to cool and trimmed. Slices with 5 μm thickness were obtained from the trimmed wax blocks with a slicer, and transferred from the sheeter to a glass slide, and dewaxed and rehydrated. The slices were stained with alizarin red staining solution for 3 minutes, washed with water, dried and permeabilized with xylene for 5 minutes. The slices were sealed with neutral gum and images of the staining results were collected through a pathology slide scanner.

The biological valve materials of sample 1, sample 2, sample 8 and Control Group 1 (glutaraldehyde crosslinked porcine pericardium) were cut into 1*1 cm² sheets for anti-calcification tests.

TABLE 5
Calcium content of biological valve materials after
subcutaneous implantation in rats for 30 days

| Sample | Calcium content (mg/g) |
|---|---|
| Control Group 1 (glutaraldehyde | 73.8 ± 11.3 |
| crosslinked porcine pericardium) | |
| Sample 1 | 21.3 ± 2.7 |
| Sample 2 | 14.1 ± 5.4 |
| Sample 8 | 5.9 ± 0.87 |

The calcium contents of sample 1, sample 2, sample 8 and Control Group 1 (glutaraldehyde crosslinked porcine pericardium) after being implanted subcutaneously in rats for 30 days was measured to characterize the degree of calcification of samples. As shown in Table 5, the calcium contents of sample 1, sample 2, sample 8 and Control Group 1 after being implanted subcutaneously in rats for 30 days were all lower than that of the Control Group 1 (glutaraldehyde crosslinked porcine pericardium). This result indicates that the method for preparing biological valve materials by post-double bond functionalized copolymerization and crosslinking can improve the anti-calcification performance of biological valves.

### Alizarin red Staining Experiment:

Sample 1, sample 2, sample 8 and Control Group 1 were implanted into the subcutaneous tissue of rats for 30 days and then taken out and fixed with paraformaldehyde tissue fixative. After fixation, the samples were taken out and smoothed with a scalpel, and then transferred to a dehydration box. The material samples were dehydrated using gradients of 50%, 75%, 85%, 95% (v/v) and absolute ethanol. After dehydration, the material samples were transferred to an embedding machine and embedded in melted wax, and then transferred to a −20° C. refrigerator to cool and trimmed. Slices with 3 to 5 μm thickness were obtained from the trimmed wax blocks with a slicer, and transferred from the sheeter to a glass slide, and dewaxed and rehydrated. The slices were stained with alizarin red staining solution for 3 minutes, washed with water, dried and permeabilized with xylene for 5 minutes. The slices were sealed with neutral gum and images of the staining results were collected through a pathology slide scanner.

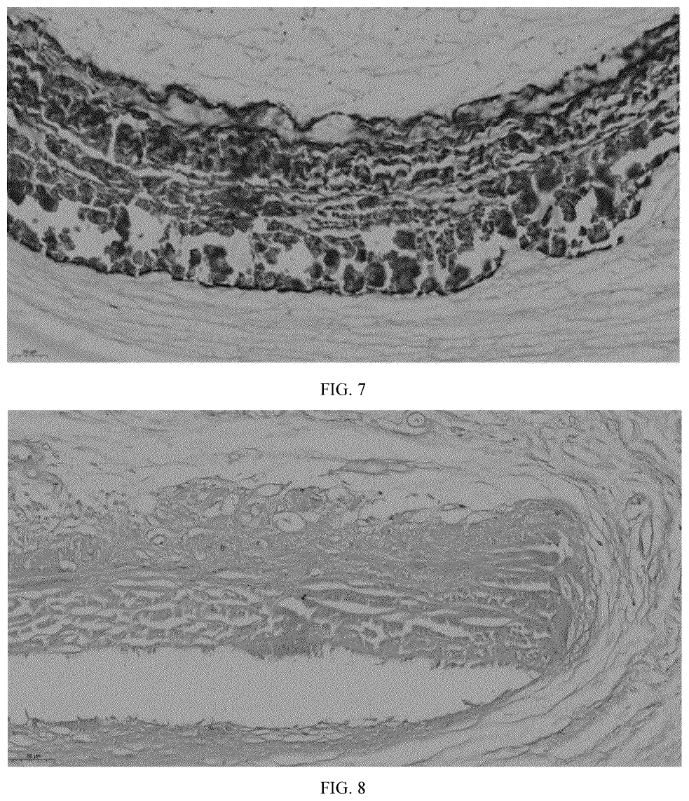

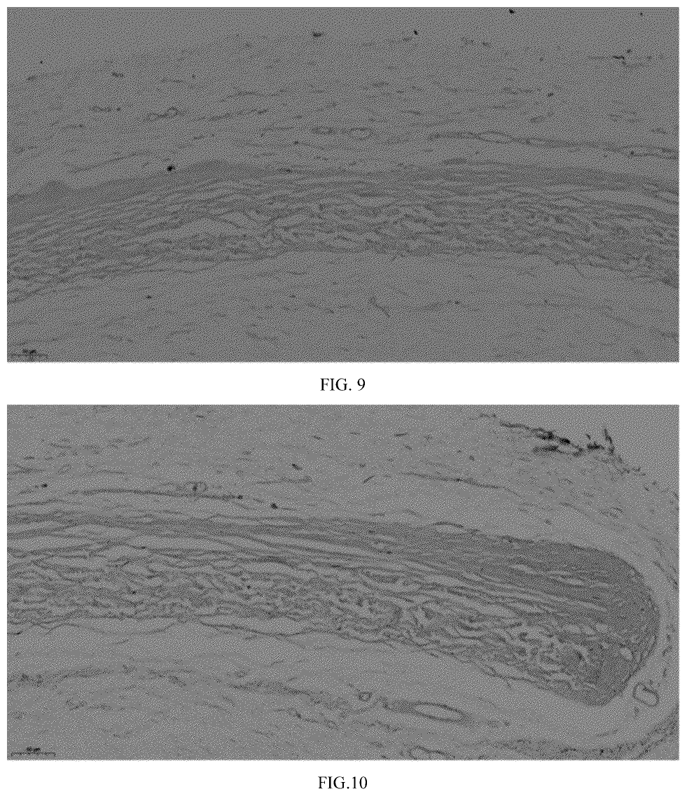

The alizarin red staining experiment was conducted on Control Group 1 (glutaraldehyde crosslinked porcine pericardium), sample 1, sample 2, and sample 8 after being implanted subcutaneously in rats for 30 days to directly observe the degree of calcification of samples. Images of the alizarin red staining results of sample slices after being implanted subcutaneously in rats for 30 days are shown in FIGS. 7 to 10, wherein the darker the color of the sample after alizarin red staining, the higher the degree of calcification. Compared with the alizarin red staining result of slice of control sample 1 (glutaraldehyde crosslinked porcine pericardium) (FIG. 7), the color of the alizarin red staining results of slices of sample 1 (FIG. 8), sample 2 (FIG. 9), and sample 5 (FIG. 10) is obviously lighter, which directly indicates that the degrees of calcification of sample 1, sample 2, and sample 8 are lower than that of the Control Group 1, that is, compared with Control Group 1, sample 1, sample 2, and sample 8 have stronger anti-calcification effect. The alizarin red staining results of biological valve materials after being implanted subcutaneously in rats for 30 days show that the method for preparing biological valve materials by post-double bond functionalized copolymerization and crosslinking of the present disclosure can improve the anti-calcification performance of biological valves.

### Example 11

Freshly collected porcine pericardium was soaked in physiological saline and washed by shaking for 2 hours, and then soaked in a 0.25% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an isopropanol aqueous solution of 5% (v/v) glycidyl methacrylate at room temperature for double-bond modification of the glutaraldehyde crosslinked porcine pericardium. The reaction time was 72 hours, and the solvent of the double-bond solution was a 25% (v/v) ethylene glycol aqueous.

After the double-bond modification was completed, the double-bond glutaraldehyde crosslinked porcine pericardium was washed with deionized water; and then the double-bond glutaraldehyde crosslinked porcine pericardium was soaked in a 5% (v/v) N-(hydroxymethyl) acrylamide solution for 5 hours.

An initiator which included ammonium persulfate at a concentration of 20 mM and sodium bisulfite at a concentration of 10 mM was added to the above solution to further initiate polymerization between the double bonds on the double-bond glutaraldehyde crosslinked biological valve material and the double bonds of N-(hydroxymethyl) acrylamide, and after reacting at 37° C. for 12 hours, a porcine pericardium crosslinked after double-bond copolymerization was obtained. The crosslinked porcine pericardium after double bond copolymerization was soaked in a 60% isopropanol aqueous solution for 45 minutes, and then soaked in a solution containing 10% glycerol, 3% polyethylene glycol (Mn=200), and 87% ethanol at room temperature for 3 hours. Excess glycerol was removed from the surface of the porcine pericardium material, which was sterilized with ethylene oxide and recorded as Sample 11.

### Example 12

Fresh porcine pericardium was placed in PS solution containing 0.5% sodium deoxycholate (surfactant) by mass, shaken for 4 hours at room temperature, and then washed with a sodium chloride aqueous solution (i.e. physiological saline) with a mass fraction of 0.9% three times.

The porcine pericardium was immersed in 0.25% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an isopropanol aqueous solution of 5% (v/v) glycidyl methacrylate at room temperature for double-bond modification of the glutaraldehyde crosslinked porcine pericardium. The reaction time was 48 hours, and the solvent of the double-bond solution was a 20% (v/v) isopropanol aqueous solution.

After the double-bond modification was completed, the double-bond glutaraldehyde crosslinked porcine pericardium was washed with deionized water; then, the double-bond glutaraldehyde crosslinked porcine pericardium was soaked in a 3% (w/v) 2-(Methacryloyloxy)ethyl]dimethyl-(3-sulfopropyl) ammonium hydroxide solution for 2 hours.

An initiator which included ammonium persulfate at a concentration of 20 mM and sodium bisulfite at a concentration of 10 mM was added to the above solution to further initiate the polymerization between the double bonds on the double-bond glutaraldehyde crosslinked biological valve material and the double bonds of 2-(Methacryloyloxy) ethyl]dimethyl-(3-sulfopropyl)ammonium hydroxide. After reacting at 37° C. for 8 hours, a porcine pericardium crosslinked after double-bond copolymerization was obtained, which was recorded as sample 12.

The above-mentioned embodiments only express several implementation methods of the present disclosure, and the descriptions thereof are relatively specific and detailed, but they cannot be understood as limiting the scope of the invention patent. It should be pointed out that, for a person of ordinary skill in the art, several variations and improvements can be made without departing from the concept of the present disclosure, and these all belong to the protection scope of the present disclosure. Therefore, the protection scope of the patent of the present disclosure shall be subject to the attached claims.

What is claimed is:

1. A method for preparing a biological valve material by copolymerization and crosslinking, comprising:
Step S110: contacting a biomaterial with an aldehyde group crosslinking agent solution for crosslinking;
Step S120: soaking the biomaterial treated in step S110 in a solution containing a first functional monomer for a chemical reaction to introduce a first carbon-carbon double bond, wherein the first functional monomer has the first carbon-carbon double bond and an ethylene oxide group;
Step S130: soaking the biomaterial treated in step S120 in a solution containing a second functional monomer, wherein the second functional monomer has a second carbon-carbon double bond and a functional group B;
Step S200, performing polymerization of carbon-carbon double bonds under an action of an initiator to obtain the biological valve material.

2. The method according to claim 1, wherein the aldehyde group crosslinking agent is glutaraldehyde or formaldehyde, the biomaterial is an animal tissue selected from one or more of the following: pericardium, valve, intestinal valve, meninges, lung valve, blood vessel, skin or ligament, and the animal tissue is a fresh animal tissue or a decellularized biological tissue.

3. The method according to claim 1, wherein the step S200 comprises:
adding the initiator to a system treated in a previous step; or
taking the biomaterial treated in the previous step out and directly or after washing soaking the biomaterial treated in the previous step in a solution containing the initiator,
wherein the initiator is a single initiator or a mixed initiator, and a polymerization reaction time is a range of 3 to 24 h.

4. The method according to claim 3, wherein the mixed initiator is:
a mixture of ammonium persulfate and sodium bisulfite, or a mixture of ammonium persulfate and sodium sulfite, or a mixture of sodium persulfate and sodium sulfite, or a mixture of potassium persulfate and sodium sulfite, or a mixture of sodium persulfate and sodium bisulfite, or a mixture of potassium persulfate and sodium bisulfite, or a mixture of potassium persulfate and tetramethylethylenediamine, or a mixture of ammonium persulfate and tetramethylethylenediamine, or a mixture of sodium persulfate and tetramethylethylenediamine; and a concentration of each component in the mixture is in a range of 1 mM to 100 mM.

5. The method according to claim 4, wherein the single initiator is any one component of the mixed initiator.

6. The method according to claim 1, wherein the first functional monomer is at least one selected from a group consisting of allyl glycidyl ether, glycidyl methacrylate and glycidyl acrylate.

7. The method according to claim 1, wherein in step S110:
a w/w concentration of the aldehyde group crosslinking agent solution is in a range of 0.1% to 5%, and a crosslinking time is in a range of 0.5 h to 120 h.

8. The method according to claim 1, wherein in step S120:
a w/w concentration of the first functional monomer in the solution containing the first functional monomer is in a range of 1% to 10%, a reaction time is in a range of 2 h to 120 h, and the solution containing the first functional monomer only contains the first functional monomer and a solvent that does not participate in chemical reaction.

9. The method according to claim 1, wherein the solvent in the solution containing the first functional monomer is one or more of the following: water, physiological saline, a neutral pH buffer, and an aqueous solution of any one of methanol, ethanol, ethylene glycol, propanol, 1,2-propanediol, 1,3-propanediol, isopropanol, butanol, isobutanol, 1,2-butanediol, 1,3-butanediol, 1,4-butanediol, and glycerol.

10. The method according to claim 1, wherein the second functional monomer is selected from one or more of the following: polyethylene glycol diacrylate, 1,4-butanediol diacrylate, ethane-1,2-diyl diacrylate, ethyl acrylate, N-methyl-2-acrylamide, N-2,2-propenyl-2-acrylamide, N-ethylacrylamide, N,N′-vinylbisacrylamide, (ethane-1,2-diylbis(oxy))bis(ethane-2,1-diyl) diacrylate, N,N′-dimethylacrylamide, N,N-dimethylmethacrylamide, and double-bond polylysine.

11. The method according to claim 1, wherein in step S130, a v/v concentration of the second functional monomer in the solution containing the second functional monomer is in a range of 0.1% to 20%; and an immersion time is in a range of 0.5 h to 120 h.

12. The method according to claim 1, wherein a v/v concentration of the second functional monomer in the solution containing the second functional monomer is a range of 0.1% to 6%.

13. The method according to claim 1, wherein the second functional monomer permeates the biomaterial by physical penetration, and the solution containing the second functional monomer only contains the second functional monomer and a solvent that does not participate in the reaction.

14. The method according to claim 1, wherein the solvent in the solution containing the second functional monomer is one or a mixture of the following: water, physiological saline, ethanol, isopropanol or a neutral pH buffer solution.

15. The method according to claim 1, wherein the functional group B is selected from at least one of the following: hydroxyl group, carboxyl group, choline carboxylate, choline sulfonate, choline phosphate, pyrrolidone, sulfonate group, carboxylate ion, sulfonate ester, sulfoxide, amide group, and methoxy group.

16. The method according to claim 1, wherein the second functional monomer is one or more selected from the following: acrylamide, acrylic acid, sodium acrylate, methacrylic acid, sodium methacrylate, 2-(prop-2-enoylamino) acetic acid, 2-acrylamido-2-methylpropanesulfonic acid, hydroxy ethyl methacrylate, 3-[2-(methacryloyloxy) ethyl]dimethylammonium] propionate, N-methyl-2-acrylamide, N-isopropylacrylamide, N-(hydroxymethyl) acrylamide, N-(2-hydroxyethyl) methacrylamide, 2-(Methacryloyloxy) ethyl]dimethyl-(3-sulfopropyl) ammonium hydroxide, 2-methacryloyloyloxyethylphosphorylcholine, N-(2-hydroxyethyl) acrylamide, N-(methoxymethoxy)methylacrylamide, 2-acrylamido-2-methylpropanesulfonic acid, and double-bond grafted hyaluronic acid.

17. A biological valve material prepared by the method according to claim 1.

18. A biological valve, comprising a stent and leaflets, wherein the leaflets are made of the biological valve material according to claim 17.

19. The biological valve according to claim 18, wherein the biological valve is a prosthetic heart valve.

20. An interventional system, comprising a prosthetic heart valve and a catheter assembly, wherein the prosthetic heart valve is delivered by the catheter assembly after being folded, and wherein the prosthetic heart valve comprises a stent and leaflets, and the leaflets are made of the biological valve material according to claim 17.
